(19) United States
(12) Patent Application Publication
(10) Pub. No.: US 2011/0142829 A1
(43) Pub. Date: Jun. 16, 2011

(54) ANTI-TUMOUR COMPOSITIONS AND METHODS

(76) Inventor: Patrick Prendergast, Byrock (AU)

(21) Appl. No.: 12/997,952

(22) PCT Filed: Jun. 18, 2009
(86) PCT No.: PCT/EP2009/057617

§ 371 Date: Feb. 15, 2011

Related U.S. Application Data
(60) Provisional application No. 61/132,385, filed on Jun. 18, 2008.

(30) Foreign Application Priority Data
Jun. 18, 2008 (GB) 0811175.9

(51) Int. Cl. A61K 39/395; A61K 31/497; A61K 33/24; A61K 31/555; A61P 35/00

(52) U.S. Cl. 424/133.1; 514/255.05; 424/649; 514/186; 514/185; 424/155.1

(57) ABSTRACT

A method for the prophylaxis and/or treatment of a tumour in a subject is provided. The method comprises the steps of administering a therapeutically effective amount of a composition comprising an anti-tumour agent and a therapeutically effective amount of a composition comprising 5-[2-pyrazinyl]-4-methyl-1,2-3-thione, or an analogue, derivative, metabolite, prodrug, solvate or pharmaceutically acceptable salt thereof to the subject. Examples of anti-tumour agents for use in the method of the invention are platinum-based agents, such as cisplatin, and monoclonal antibodies, such Cetuximab. Also provided are compositions comprising an anti-tumour agent and 5-[2-pyrazinyl]-4-methyl-1,2-3-thione, or an analogue, derivative, metabolite, prodrug, solvate or pharmaceutically acceptable salt thereof.

### FIELD OF THE INVENTION

The present invention provides compositions for use in the treatment of tumours and cancers. Also provided are methods for the treatment, suppression and amelioration of tumours and for the improved treatment of cancers using the compositions of the invention.

### BACKGROUND TO THE INVENTION

Each year 10.9 million people worldwide are diagnosed with cancer and there are 6.7 million cancer deaths. There is clearly an urgent need for improved therapeutic cancer strategies. Current methods of treatment include the use of anti-tumour agents to prevent and/or reduce tumour growth.

There are a number of possible mechanisms of action by which anti-tumour agents may act. These include, but are not limited to, inhibition of angiogenesis, inhibition of DNA synthesis, regulation of DNA/RNA transcription, inhibition of enzymes, gene regulation, inhibition of microtubule assembly, intercalation of DNA, induction of apoptosis and induction of cross-linking of DNA. Some anti-tumour agents involve more than one mechanism of action. For example, in certain cases the induction of cross-linking of DNA may result in apoptosis. Monoclonal antibodies may be used to specifically target cancer cells for destruction by recognising and binding to receptors found on cancer cells.

In some cases, the use of anti-tumour agents is limited due to the toxicity of the anti-tumour agents. This toxicity may be linked to the dose of the anti-tumour agent administered. Accordingly, it would be beneficial if the dose of the anti-tumour agent could be reduced without a corresponding loss in effectiveness of the treatment.

Cisplatin (also known as cisplatinum or cis-diamminedichloridoplatinum(II) (CDDP)) is one example of a commonly used anti-tumour agent. Cisplatin is a platinum-based chemotherapy drug. Platinum complexes are formed in cells that bind and cause cross-linking of DNA leading to apoptosis. Cisplatin may be used to treat various types of cancers, including sarcomas, some carcinomas (e.g. small cell lung cancer, and ovarian cancer), lymphomas and germ cell tumours. Other members of the same class include carboplatin and oxaliplatin. Cisplatin has a number of known side effects which can limit its use in the treatment of cancer. The main side effects include nephrotoxicity, neurotoxicity, nausea and vomiting, ototoxicity, alopecia and electrolyte disturbance.

In certain cases, Cetuximab (marketed under the name Erbitux) may be administered to cancer patients who can no longer be treated with cisplatin due to side effects, primarily nephrotoxicity. Cetuximab is a chimeric monoclonal antibody which is believed to bind the extracellular domain of the epidermal growth factor receptor (EGFR), thus preventing the binding of ligands to the EGFR. This blocks activation of the EGFR, which in turn blocks downstream signalling and results in impaired cell growth and proliferation. Cetuximab is also thought to mediate antibody-dependent cellular cytotoxicity (ADCC). Cetuximab may be administered by intravenous injection for treatment of metastatic colectoral cancer and head and neck cancer. Possible side effects include allergic reactions, skin changes, breathlessness, nausea and vomiting, diarrhoea, fever and conjunctivitis.

Despite the wide variety of chemotherapeutic agents used for the treatment and suppression of tumours, a need remains for improved anti-tumour compositions preferably having reduced side effects.

### SUMMARY OF THE INVENTION

The inventor of the present invention has determined that when oltipraz (5-[2-pyrazinyl]-4-methyl-1,2-3-thione) is administered with other known anti-tumour agents, such as platinum-based chemotherapy drugs, including cisplatin, or monoclonal antibodies, including Cetuximab, improved therapeutic effects are observed with an increased reduction in tumour growth. This determination may advantageously be used to provide improved compositions and methods for the treatment of tumours and cancer. Further, this determination may be used to reduce the required treatment dose of an anti-tumour agent, such as cisplatin or Cetuximab, as, when an anti-tumour agent is administered in combination with oltipraz, the same therapeutic effect may be obtained using a lower dose as compared to the therapeutic effect obtained when the anti-tumour agent is administered alone at a higher dose. This may reduce the side effects, such as nephrotoxicity, which often occur with anti-tumour treatments.

According to a first aspect of the present invention there is provided a composition comprising an anti-tumour agent and 5-[2-pyrazinyl]-4-methyl-1,2-3-thione, or an analogue, derivative, metabolite, prodrug, solvate or pharmaceutically acceptable salt thereof.

According to a second aspect of the present invention there is provided a pharmaceutical composition comprising an anti-tumour agent and 5-[2-pyrazinyl]-4-methyl-1,2-3-thione, or an analogue, derivative, metabolite, prodrug, solvate or pharmaceutically acceptable salt thereof, and at least one pharmaceutically acceptable carrier, excipient or diluent.

According to a third aspect of the present invention there is provided a composition comprising an anti-tumour agent and 5-[2-pyrazinyl]-4-methyl-1,2-3-thione, or an analogue, derivative, metabolite, prodrug, solvate or pharmaceutically acceptable salt thereof, for use as a medicament.

According to a fourth aspect of the present invention there is provided a composition comprising an anti-tumour agent and 5-[2-pyrazinyl]-4-methyl-1,2-3-thione, or an analogue, derivative, metabolite, prodrug, solvate or pharmaceutically acceptable salt thereof, for use in the prevention and/or treatment of a tumour.

According to a fifth aspect of the present invention there is provided the use of a composition comprising an anti-tumour agent and 5-[2-pyrazinyl]-4-methyl-1,2-3-thione, or an analogue, derivative, metabolite, prodrug, solvate or pharmaceutically acceptable salt thereof, in the preparation of a medicament for the treatment and/or prophylaxis of a tumour.

According to a sixth aspect of the present invention there is provided a method for the prophylaxis and/or treatment of a tumour in a subject, the method comprising the step of:
administering a therapeutically effective amount of a composition comprising an anti-tumour agent and 542-pyrazinyl]-4-methyl-1,2-3-thione, or an analogue, derivative, metabolite, prodrug, solvate or pharmaceutically acceptable salt thereof, to the subject.

According to a further aspect of the present invention there is provided a method for the prophylaxis and/or treatment of a tumour in a subject, the method comprising the steps of:
administering a therapeutically effective amount of a composition comprising an anti-tumour agent; and
administering a therapeutically effective amount of a composition comprising 5-[2-pyrazinyl]-4-methyl-1,2-3-thione, or an analogue, derivative, metabolite, prodrug, solvate or pharmaceutically acceptable salt thereof to the subject.

According to a further aspect of the present invention there is provided a composition comprising 5-[2-pyrazinyl]-4-methyl-1,2-3-thione, or an analogue, derivative, metabolite, prodrug, solvate or pharmaceutically acceptable salt thereof, for use in the reduction of the toxicity of an anti-tumour agent.

According to a further aspect of the present invention there is provided a composition comprising 5-[2-pyrazinyl]-4-methyl-1,2-3-thione, or an analogue, derivative, metabolite, prodrug, solvate or pharmaceutically acceptable salt thereof, for use in the reduction of the side effects of treatment with an anti-tumour agent.

According to a further aspect of the present invention there is provided the use of a composition comprising 5-[2-pyrazinyl]-4-methyl-1,2-3-thione, or an analogue, derivative, metabolite, prodrug, solvate or pharmaceutically acceptable salt thereof, in the preparation of a medicament for reducing the toxicity of an anti-tumour agent.

According to a further aspect of the present invention there is provided the use of a composition comprising 5-[2-pyrazinyl]-4-methyl-1,2-3-thione, or an analogue, derivative, metabolite, prodrug, solvate or pharmaceutically acceptable salt thereof, in the preparation of a medicament for reducing the side effects of treatment with an anti-tumour agent.

According to a further aspect of the present invention there is provided a method for reducing the toxicity of an anti-tumour agent in a subject, the method comprising the steps of:
administering a therapeutically effective amount of a composition comprising 5-[2-pyrazinyl]-4-methyl-1,2-3-thione, or an analogue, derivative, metabolite, prodrug, solvate or pharmaceutically acceptable salt thereof; and
administering a therapeutically effective amount of a composition comprising the anti-tumour agent to the subject.

According to a further aspect of the present invention there is provided a method for reducing the side effects of treatment of a subject with an anti-tumour agent, the method comprising the steps of:

administering a therapeutically effective amount of a composition comprising 5-[2-pyrazinyl]-4-methyl-1,2-3-thione, or an analogue, derivative, metabolite, prodrug, solvate or pharmaceutically acceptable salt thereof; and
administering a therapeutically effective amount of a composition comprising the anti-tumour agent to the subject.

According to a further aspect of the present invention there is provided a method for reducing the toxicity of an anti-tumour agent in a subject, the method comprising the steps of:
administering a therapeutically effective amount of a composition comprising the anti-tumour agent and 5-[2-pyrazinyl]-4-methyl-1,2-3-thione, or an analogue, derivative, metabolite, prodrug, solvate or pharmaceutically acceptable salt thereof, to the subject.

According to a further aspect of the present invention there is provided a method for reducing the side effects of an anti-tumour agent in a subject, the method comprising the steps of:
administering a therapeutically effective amount of a composition comprising the anti-tumour agent and 5-[2-pyrazinyl]-4-methyl-1,2-3-thione, or an analogue, derivative, metabolite, prodrug, solvate or pharmaceutically acceptable salt thereof, to the subject.

According to a further aspect of the present invention there is provided a combined medicament comprising at least one anti-tumour agent and 5-[2-pyrazinyl]-4-methyl-1,2-3-thione or an analogue, derivative, metabolite, prodrug, solvate or pharmaceutically acceptable salt thereof.

According to a further aspect of the present invention there is provided a combination therapy comprising two separate pharmaceutical preparations, a first pharmaceutical preparation comprising 5-[2-pyrazinyl]-4-methyl-1,2-3-thione, or an analogue, derivative, metabolite, prodrug, solvate or pharmaceutically acceptable salt thereof and a second pharmaceutical preparation comprising an anti-tumour agent wherein the first and second pharmaceutical preparations can be administered to a subject in need thereof simultaneously, semi-simultaneously, sequentially, separately or spaced out over time, for example on alternate days.

The invention further provides kits for carrying out the therapeutic regimens of the invention. Such kits may comprise, in one or more containers, therapeutically effective amounts of an anti-tumour agent and 5-[2-pyrazinyl]-4-methyl-1,2-3-thione, or an analogue, derivative, metabolite, prodrug, solvate or pharmaceutically acceptable salt thereof, in a pharmaceutically acceptable form. Such kits may further include instructions for use in the performance of the methods of the invention, or may provide further information to provide a physician with information appropriate to treating tumours and/or cancer.

Preferred features of each aspect of the invention are discussed below. Preferred features of each aspect of the invention are as for each of the other aspects mutatis mutandis.

### BRIEF DESCRIPTION OF THE DRAWINGS

FIG. 1 shows the mean daily percentage weight change for control, cisplatin alone, oltipraz alone and cisplatin plus oltipraz treatment groups (error bars represent the SEM);

FIG. 2 shows the mean area under the curve (AUC) data for the percent weight change exhibited by the control, cisplatin alone, oltipraz alone and cisplatin plus oltipraz treatment groups;

FIG. 3 shows the mean tumour volumes exhibited by the control, cisplatin alone, oltipraz alone and cisplatin plus oltipraz treatment groups calculated from tumour length and width measurements (error bars represent the SEM);

FIG. 4 shows the mean area under the curve (AUC) data for the changes in tumour volume exhibited by the control, cisplatin alone, oltipraz alone and cisplatin plus oltipraz treatment groups;

FIG. 5 shows the mean daily percentage weight change for control, Cetuximab alone and Cetuximab plus oltipraz treatment groups (error bars represent the SEM);

FIG. 6 shows the mean area under the curve (AUC) data for the percent weight change exhibited by the control, Cetuximab alone and Cetuximab plus oltipraz treatment groups;

FIG. 7 shows the mean tumour volumes exhibited by the control, Cetuximab alone and Cetuximab plus oltipraz treatment groups calculated from tumour length and width measurements (error bars represent the SEM); and

FIG. 8 shows the mean area under the curve (AUC) data for the changes in tumour volume exhibited by the control, Cetuximab alone and Cetuximab plus oltipraz treatment groups.

### DETAILED DESCRIPTION OF THE INVENTION

The present invention provides an anti-tumour composition, for example, a chemotherapeutic drug for use in the treatment and/or prophylaxis of cancer comprising two types of active ingredients, namely 5-[2-pyrazinyl]-4-methyl-1,2-3-thione, or an analogue, derivative, metabolite, prodrug, solvate or pharmaceutically acceptable salt thereof, and an anti-tumour agent that can be administered simultaneously, semi-simultaneously, separately or sequentially. The anti-tumour composition of the invention may be used to reduce the volume of a tumour and/or the rate of tumour growth. The anti-tumour composition of the invention may further be used to reduce the side effects and/or toxicity of the anti-tumour agent of the composition.

The term “anti-tumour agent” as used herein is understood to refer to any agent other than oltipraz that is used in the treatment and/or prophylaxis of tumours and/or cancer. Preferably the agent is effective in the treatment and/or prophylaxis of tumours and/or cancer. The agent may be used to reduce and/or prevent the formation and/or growth of tumours, in particular malignant tumours. Tumour volume and/or the rate of growth of the tumours may be reduced. The term “anti-tumour agent” as used herein is intended to include chemotherapeutic agents and agents used in immunotherapy, for example monoclonal antibodies.

The term “tumour” as used herein is intended to include both benign and malignant tumours. The term “cancer” as used herein is understood to refer to all cancer types and to include malignant tumours.

The terms “oltipraz” and “5-[2-pyrazinyl]-4-methyl-1,2-3-thione” are used interchangeably herein. The structure of 5-[2-pyrazinyl]-4-methyl-1,2-3-thione (also known as 4-methyl-5(2-pyrazinyl)-3H-1,2-dithiole-3-thione or 5-(2-pyrazinyl)-4-methyl-1,2-dithiol-3-thione) is shown below as Formula 1.

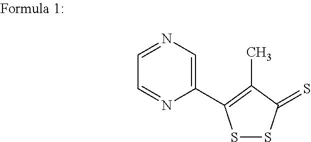

Pharmaceutically acceptable salts are salts that retain the desired biological activity of the parent compound and do not impart undesired toxicological effects. Examples of pharmaceutically acceptable salts are discussed in Berge et al., 1977, “Pharmaceutically Acceptable Salts,” J. Pharm. ScL, Vol. 66, pp. 1-19.

The term “solvate” is used herein in the conventional sense to refer to a complex of solute (e.g., active compound or salt of active compound) and solvent. If the solvent is water, the solvate may be conveniently referred to as a hydrate, for example, a hemihydrate, monohydrate, dihydrate, trihydrate, tetrahydrate and the like.

The term “prodrug” is used herein to refer to a compound which can convert to the biologically active compound by metabolism or hydrolysis. A prodrug can be made using pharmacological techniques known to those skilled in the art.

Metabolites may result from the metabolism, for example by molecular rearrangement, or hydrolysis of oltipraz following administration to a subject.

The present invention is further intended to encompass the use of derivatives and analogues of oltipraz. In this context, derivatives or homologues are molecules having substantial structural similarities to oltipraz and analogues are molecules having substantial biological similarities regardless of structural similarities. Preferably the analogue or derivative will retain the appropriate functional activity of oltipraz, though not necessarily to the same degree as oltipraz.

As used herein, the term “subject” or “patient” refers to an animal, preferably a mammal, and in particular a human.

As used herein, the term “therapeutically effective amount” means the amount of a composition which is sufficient to show benefit to the subject. In particular, the benefit may be the treatment, partial treatment or amelioration of at least one symptom associated with cancer and/or a tumour. In the case of prophylaxis, the benefit may be to prevent or suppress the initial onset, progression or recurrence of cancer and/or a tumour, or at least one symptom thereof. The benefit includes a reduction in the rate of tumour formation and/or in the size of a tumour, in particular a malignant tumour. The benefit further includes a reduction in the side effects and/or toxicity associated with treatment with an anti-tumour agent.

The terms “side effect” and “toxicity” as used herein refer to unwanted effects of the treatment of a subject with an anti-tumour agent. The effects are caused by the treatment of the subject with the anti-tumour agent, but are not beneficial to the subject.

The term “reducing” or “reduction” as used herein to refer to the reduction of side effects and/or toxicity of an anti-tumour agent observed when the agent is combined with oltipraz is intended to encompass both prevention and lessening of the side effects. It includes lessening of the severity of the side effects and the progression and onset of the side effects. It also includes a reduction in the number of side effects.

In certain embodiments of the above mentioned aspects of the invention, the metabolite of 5-[2-pyrazinyl]-4-methyl-1,2-3-thione is the pyrrolopyrazine derivative metabolite 3, also known as M3.

In certain embodiments, the analogue of 5-[2-pyrazinyl]-4-methyl-1,2-3-thione is a compound of the 1,2-dithiol-3-thione class, for example, anethole trithione ((5-(p-methoxyphenyl)-3H-1,2-dithiole-3-thio) (also known as anetol tritiona or SONICUR™)

In certain embodiments, the 5-[2-pyrazinyl]-4-methyl-1,2-3-thione analogue is 1,2-dithiole-3-thione (D3T) or an analogue thereof. Typically the 1,2-dithiole-3-thione analogue has the following formula:

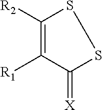

wherein:
in the case of 5-substituted analogues:
   R₁ is H, R₂ is phenyl and X is S,
   R₁ is H, R₂ is 4-methoxyphenyl and X is S,
   R₁ is H, R₂ is 2-pyrazinyl and X is O or
   R₁ is H, R₂ is 2-(5,6-dimethyl)pyrazinyl and X is S;
in the case of 5-substituted-4-methyl analogues:
   R₁ is CH₃, R₂ is 2-pyridyl and X is S,
   R₁ is CH₃, R₂ is 3-pyridyl and X is S,
   R₁ is CH₃, R₂ is 4-pyridyl and X is S,
   R₁ is CH₃, R₂ is 3-pyridazinyl and X is S,
   R₁ is CH₃, R₂ is 2-thiofuranyl and X is S or
   R₁ is CH₃, R₂ is 2-(2-pyrazinyl)ethylene and X is S;
in the case of 4-substituted-5-(2-pyrazinyl) analogues:
   R₁ is CH₃, R₂ is 2-pyrazinyl and X is S,
   R₁ is CH₃, R₂ is 2-pyrazinyl and X is O,
   R₁ is CH₂OH, R₂ is 2-pyrazinyl and X is S,
   R₁ is CH₂CH₃, R₂ is 2-pyrazinyl and X is S or
   R₁ is (CH₂)₃CH₃, R₂ is 2-pyrazinyl and X is S;
in the case of miscellaneous analogues:
   R₁ is CO₂C₂H₅, R₂ is 2-pyridyl and X is S,
   R₁ is CO₂C₂H₅, R₂ is 4-pyridyl and X is S,
   R₁ is C₁, R₂ is [4-(2-propyl)phenyl]amino and X is S,
   R₁ is C₁, R₂ is [4-(2-propyl)phenyl]amino and X is O,
   R₁ is CH₂CO₂C₂H₅, R₂ is 5-pyrimidyl and X is S,
   R₁ is CH₂CON[CH(CH₃)₂]₂, R₂ is 5-pyrimidyl and X is S,
   R₁ is phenethyl, R₂ is 3-pyridazinyl and X is S,
   R₁ is H, R₂ is 4-pyridyl and X is N—O—(CH₂)₃N(CH₃)₂ or
   R₁ is (CH₂)₃CH₃, R₂ is 3-(6-dimethylamino)pyridazinyl and X is S.

In certain further embodiments, R₁ is fluorine or bromine, and R₂ and X are selected from the substituents listed above.

In certain embodiments, the composition of the invention is co-administered along with, or formulated with, carboxymethyl cellulose (CMC). In certain embodiments, 5-[2-pyrazinyl]-4-methyl-1,2-3-thione, or the analogue, derivative, metabolite, prodrug, solvate or pharmaceutically acceptable salt thereof, is formulated in carboxymethyl cellulose (CMC).

The inventor has surprisingly identified that administering 5-[2-pyrazinyl]-4-methyl-1,2-3-thione along with carboxymethyl cellulose results in a marked reduction in toxicity associated with 5-[2-pyrazinyl]-4-methyl-1,2-3-thione administration. Carboxymethyl cellulose (CMC) is a cellulose derivative with carboxymethyl groups bound to some of the hydroxyl groups of the glucopyranose monomers that make up the cellulose backbone.

Specifically, the inventor has identified that when formulated with CMC, 5-[2-pyrazinyl]-4-methyl-1,2-3-thione can be administered to a subject in an amount of up to 2000 mg/kg without significant toxicity resulting. Without wishing to be bound by theory, it is hypothesised that when formulated with carboxymethyl cellulose, 5-[2-pyrazinyl]-4-methyl-1,2-3-thione is not absorbed into the bloodstream but becomes associated with the outer wall of the digestive tract, this resulting in an effective lining of the digestive tract, which serves to protect against damage, such as gastrointestinal damage.

In certain embodiments, the 5-[2-pyrazinyl]-4-methyl-1,2-3-thione compound is administered with, or formulated with, a sulphur-containing amino acid such as cysteine or an analogue, derivative, salt or solvate thereof.

Oral administration of 5-[2-pyrazinyl]-4-methyl-1,2-3-thione with cysteine has been shown to result in a marked increase in both the extent and rate of 5-[2-pyrazinyl]-4-methyl-1,2-3-thione bioavailability (Hassan M. Ali et al., 1984; Chemotherapy 30: 255-261).

In certain embodiments of the invention, the anti-tumour agent is a platinium-based agent. In other embodiments, the anti-tumour agent is a monoclonal antibody. Typically, the monoclonal antibody has binding specificity to epidermal growth factor receptors or EGFRs. In certain embodiments, the anti-tumour agent mediates antibody-dependent cellular cytotoxicity (ADCC). In certain embodiments, the anti-tumour agent is cisplatin. In other embodiments, the anti-tumour agent is Cetuximab.

In certain embodiments, the anti-tumour agent is selected from the group comprising, but not limited to, angiogenesis inhibitors, DNA synthesis inhibitors, DNA/RNA transcription inhibitors, enzyme inhibitors, gene regulators, microtubule assembly inhibitors, DNA intercalators, apoptosis inducers and DNA cross-linkers.

In still other embodiments, the anti-tumour agent is selected from the group comprising, but not limited to, dexamethasone; mitotic inhibitors, such as vinblastine; alkylating agents, such as carboplatin and cyclophosphamide; inhibitors of microtubule assembly, such as paclitaxel or other taxanes; anti-metabolites, such as 5-fluorouracil, capecitabine, cytosine arabinoside and hydroxyurea; intercalating antibiotics, such as, adriamycin and bleomycin; immunostimulants, such as trastuzumab; DNA synthesis inhibitors, such as, gemcitabine; enzymes, such as asparaginase; topoisomerase inhibitors, such as etoposide; biological response modifiers, such as interferon; and anti-hormones, for example, antioestrogens, such as tamoxifen, or antiandrogens, such as (4′-cyano-3-(4-fluorophenylsulphonyl)-2-hydroxy-2-methyl-3′-(trifluoromethyl)-propionanilide and other therapeutic agents and principles as described in, for example, DeVita, V. T., Jr., Hellmann, S., Rosenberg, S. A.; in: Cancer: Principles & Practice of Oncology, 5th ed., Lippincott-Raven Publishers (1997).

In certain embodiments, the use of 5-[2-pyrazinyl]-4-methyl-1,2-3-thione, or an analogue, derivative, metabolite, prodrug, solvate or pharmaceutically acceptable salt thereof, allows a lower dose of the anti-tumour agent to be used without a corresponding reduction in the observed therapeutic effect. In certain embodiments, the use of 5-[2-pyrazinyl]-4-methyl-1,2-3-thione, or an analogue, derivative, metabolite, prodrug, solvate or pharmaceutically acceptable salt thereof, reduces or limits side effects caused by the anti-tumour agent. Without being bound by theory, this reduction may be due to the reduced dosage of the anti-tumour agent.

In certain embodiments, the combination of 5-[2-pyrazinyl]-4-methyl-1,2-3-thione, or an analogue, derivative, metabolite, prodrug, solvate or pharmaceutically acceptable salt thereof, with the anti-tumour agent results in a synergistic effect. In certain embodiments, the synergistic effect leads to a reduction in tumour volume which is greater than the reduction in tumour volume observed when 5-[2-pyrazinyl]-4-methyl-1,2-3-thione, or an analogue, derivative, metabolite, prodrug, solvate or pharmaceutically acceptable salt thereof, or the anti-tumour agent is administered alone at the same dose. In certain embodiments, the synergistic effect leads to a reduction in toxicity and/or side effects of the anti-tumour agent wherein the toxicity and/or side effects of the anti-tumour agent are less than those observed when the anti-tumour agent is administered alone at the same dose.

In certain embodiments, the anti-tumour agent and 5-[2-pyrazinyl]-4-methyl-1,2-3-thione, or an analogue, derivative, metabolite, prodrug, solvate or pharmaceutically acceptable salt thereof, are provided sequentially, simultaneously or separately. Said compounds may be in the same or different forms, for example a solid and a liquid, and may be administered by the same or different routes. Where the compounds are administered sequentially, in certain embodiments, the anti-tumour agent may be administered first. In certain further embodiments, the anti-tumour agent may be administered following administration of 5-[2-pyrazinyl]-4-methyl-1,2-3-thione, or an analogue, derivative, metabolite, prodrug, solvate or pharmaceutically acceptable salt thereof. 5-[2-pyrazinyl]-4-methyl-1,2-3-thione, or an analogue, derivative, metabolite, prodrug, solvate or pharmaceutically acceptable salt thereof, may be re-administered following administration of the anti-tumour agent.

The concentrations of 5-[2-pyrazinyl]-4-methyl-1,2-3-thione, or an analogue, derivative, metabolite, prodrug, solvate or pharmaceutically acceptable salt thereof, and anti-tumour agents used are preferably sufficient to provide a synergistic effect. In certain embodiments, tumour growth inhibition using 5-[2-pyrazinyl]-4-methyl-1,2-3-thione, or an analogue, derivative, metabolite, prodrug, solvate or pharmaceutically acceptable salt thereof, in combination with the anti-tumour agent can be at least 65%, more preferably at least 69%, more preferably at least 75%, yet more preferably at least 80%, yet more preferably 85% and most preferably at least 89%.

In certain embodiments, the side effects and/or toxicity which are reduced are selected from one or more of the group comprising nephrotoxicity, neurotoxicity, nausea, vomiting, ototoxicity, alopecia, electrolyte disturbance, allergic reactions, skin changes, breathlessness, diarrhoea, fever and conjunctivitis, or a combination thereof.

In certain embodiments, a further anti-tumour agent may also be administered or a further cancer treatment also applied, for example, radiotherapy.

In certain embodiments, the anti-tumour agent and 5-[2-pyrazinyl]-4-methyl-1,2-3-thione, or an analogue, derivative, metabolite, prodrug, solvate or pharmaceutically acceptable salt thereof, are co-administered. Co-administration means that these compounds may be administered together as a single composition or as part of the same unitary dose or are administered separately, but as part of the same therapeutic regimen or treatment program. In certain embodiments, the compounds are administered to the subject at the same time. Where the compounds are administered separately, the co-administration of the components does not impose a restriction on the timing, frequency, dosage or order of administration of the compounds.

Suitably administration is by parenteral administration. Parenteral administration may be intravenous administration or subcutaneous administration. In further embodiments, the route of administration is rectal, for example by means of a suppository, transdermal or transmucosal.

In certain embodiments administration may be by topical application including, but not limited to, buccal and sublingual administration. Suitable formulations for topical administration include creams, gels, jellies, mucliages, pastes and ointments. In certain embodiments, the composition may be formulated for transdermal administration, for example in the form of transdermal patches. There is no specific limitation to the form of administration of an anti-tumour compound and where two anti-tumour compounds are administered each compound may have a distinct form of administration.

The tumours against which the anti-tumour agents of the invention or combinations thereof are administered encompass all tumours occurring in an animal, especially a human. In particular embodiments, the anti-tumour agents of the invention, the combinations thereof and treatment of tumours and cancer includes treatment of neoplastic growths or tumours, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumour and leukaemias, including acute lymphoblastic leukaemia and acute myeloblastic leukaemia, gliomas and retinoblastomas. In specific embodiments, the tumours can be tumours of the lung.

The compositions and methods of the invention may be particularly useful in the treatment of existing cancer and in the prevention of the recurrence of cancer after initial treatment or surgery.

The effective amount of the composition for the treatment and/or prophylaxis of a tumour and/or cancer may be provided in a single dosage regimen or a multi-dose regimen. Dosage regimens may be optimised to provide the maximum efficacy of the combination. Administration may vary in duration from a rapid administration to a continuous perfusion.

In certain embodiments oral administration may be used, for example in the form of an oral rinse, or administration to the lungs as an aerosol via oral or nasal inhalation. For administration via the oral or nasal inhalation routes, preferably the active ingredient will be in a suitable pharmaceutical formulation and may be delivered using a mechanical form including, but not restricted to, an inhaler or nebuliser device.

For intravenous injection, the active ingredient will be in the form of a parenterally acceptable aqueous solution which is pyrogen-free and has suitable pH, isotonicity and stability. Methods of preparation of suitable solutions using, for example, isotonic vehicles such as sodium chloride injection, Ringer's injection or Lactated Ringer's injection will be known to those of relevant skill in the art. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Various delivery systems are known and can be used to administer the compositions of the present invention. More specifically, the compositions may be administered via microspheres, liposomes, or other microparticulate delivery systems or sustained release formulations placed in certain tissues including blood. Suitable examples of sustained release carriers include semipermeable polymer matrices in the form of shared articles, for example suppositories or microcapsules. Implantable or microcapsular sustained release matrices such as polylactides are also provided.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7th Edition ISBN 0-683305-72-7, the entire disclosures of which are herein incorporated by reference.

The composition of the invention is preferably administered to an individual in a “therapeutically effective amount” as defined hereinafter. The actual amount administered in order to achieve an effect, as well as the rate and time-course of administration, will depend on, and can be determined with due reference to, the nature and severity of the condition which is being treated, as well as factors such as the age, sex, weight of the patient to be treated and the route of administration. Toxicity and efficacy of the compositions can be determined by standard pharmaceutical procedures.

Unless otherwise defined, all technical and scientific terms used herein have the meaning commonly understood by a person who is skilled in the art in the field of the present invention.

The compounds disclosed herein extend to other forms of said compounds, said other forms including the well known ionic, salt, solvate, and protected forms of these substituents. For example, a reference to carboxylic acid (—COOH) also includes the anionic (carboxylate) form (—COO), a salt or solvate thereof, as well as conventional protected forms. Similarly, a reference to an amino group includes conventional protected forms of an amino group. Similarly, a reference to a hydroxyl group also includes the anionic form (—O—), a salt or solvate thereof, as well as conventional protected forms.

Certain compounds may exist in one or more particular geometric, optical, enantiomeric, diasteriomeric, epimeric, atropic, stereoisomeric, tautomeric, conformational or anomeric forms, including, but not limited to cis- and trans-forms, E- and Z-forms, c-, t- and r-forms, endo and exo-forms, R—, S— and meso forms, D- and L-forms, d- and I— forms, (+) and (−) forms, keto-, enol- and enolate-forms, syn and anti-forms, synclinal and anticlinal forms, alpha and beta forms, axial and equatorial forms, boat-, chair-, twist-, envelope-, and halfchair-forms, and combinations thereof, herein collectively referred to as “isomers” or “isomeric forms”.

Unless otherwise specified, a reference to a particular compound includes all such isomeric forms, including (wholly or partial) racemic and other mixtures thereof. Methods for the preparation (e.g. asymmetric synthesis) and separation (e.g. fractional crystallisation and chromatographic means) of such isomeric forms are either known in the art or are readily obtainable. Unless otherwise specified, a reference to a particular compound also includes ionic, salt, solvate and protected forms thereof.

The phrase “substituted” or “optionally substituted” as used herein means a parent group which may be unsubstituted or which may be substituted. Unless otherwise specified, the term “substituted” as used herein relates to a parent group which bears one or more substituents. The term “substituent” is used herein in the conventional sense and refers to a chemical moiety which is attached to, or if appropriate, fused to, a parent group. A wide variety of substituents are well known, and methods for their formation and introduction into a variety of parent groups are also well known to the person skilled in the art.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well a two or more different active agents in combination, while references to “a carrier” includes mixtures of two or more carriers as well as a single carrier, and the like. Reference to “a tumour” includes a single tumour in addition to two or more tumours of a same type or of different types.

The invention will now be described with reference to the following examples which are provided for the purpose of illustration and are not intended to be construed as being limiting on the present invention, and further, with reference to the figures.

### EXAMPLES

### Example 1

### An Evaluation of Oltipraz in Combination with Cisplatin For Efficacy in the Reduction of Tumour Growth

A study was carried out to evaluate the effects of oltipraz on tumour growth when used in conjunction with cisplatin in the A549 lung cancer model in mice. A549 was used due to its reported sensitivity to cisplatin.

### Materials and Methods

### Treatment Groups

Forty-eight female nude mice (nu/nu) were randomly assigned into four treatment groups. Each mouse was inoculated with 1×10⁶A549 lung cancer cells in a volume of 0.05 mL on their lower left flank with 0.05 mL of Matrigel. The groups were treated with vehicle (0.5% carboxymethyl cellulose (CMC)), cisplatin alone, oltipraz alone or cisplatin plus oltipraz as detailed in Table 1. Initiation of treatment was designated day 1.

TABLE 1
Treatment Groups

| | Number | Tumour | | Drug | | |
|---|---|---|---|---|---|---|
| | of | cell | Adjuvant | Treatment | | Dose |
| Group | animals | inoculum | Therapy | & Dosing | Route | Schedule |
| 1 | 12 | 1 × 10⁶ | none | 0.5% | po | qd, |
| | | | | CMC | | days 1 |
| | | | | | | and 3 |
| 2 | 12 | 1 × 10⁶ | cisplatin | 0.5% | po | qd, |
| | | | 5 mg/kg | CMC | | days 1 |
| | | | sc Day 2 | | | and 3 |
| 3 | 12 | 1 × 10⁶ | none | Oltipraz | po | qd, |
| | | | | 100 mg/kg | | days 1, |
| | | | | | | 3, 8 |
| | | | | | | and 10 |
| 4 | 12 | 1 × 10⁶ | cisplatin | Oltipraz | po | qd, |
| | | | 5 mg/kg | 100 mg/kg | | days 1 |
| | | | sc Day 2 | | | and 3 |

### Weights and Survival

All animals were weighed every day and their survival recorded in order to assess possible differences in animal weight among treatment groups as an indication of possible toxicity resulting from the treatments. Any animals exhibiting a loss of >20% of starting weight during the course of the study were euthanized.

### Tissue Culture

A549 human lung cancer cells were obtained from ATCC. These cells were grown in F-12 supplemented with 10% Fetal Calf Serum (FCS), penicillin and streptomycin, and 2 mM L-Glutamine. Cells were sub-cultured by removing the medium, rinsing twice with sterile calcium-free and magnesium-free phosphate buffered saline (PBS) and adding 1 to 2 ml of 0.25% trypsin and 0.03% EDTA solution. The flask was allowed to sit at 37° C. until the cells detached. Cells were then sub-cultured at a ratio of 1:3.

### Animals

Female Nude Mice which are homozygous for the nu gene (nu+/nu+) (Charles River Labs), aged 5 to 6 weeks at the point of tumour inoculation, with a mean pre-treatment body weight of 27.9 grams were used. Animals were individually numbered using an ear punch and housed in groups of 12 animals per cage. Animals were acclimatized prior to study commencement. During this period of at least 2 days, the animals were observed daily in order to reject animals that presented in poor condition.

### Housing

The study was performed in animal rooms provided with filtered air at a temperature of 70° F.+/−5° F. and 50%+/−20% relative humidity. Animal rooms were set to maintain a minimum of 12 to 15 air changes per hour. The room was on an automatic timer for a light/dark cycle of 12 hours on and 12 hours off with no twilight. Sterilized Bed-O-Cobs® bedding was used. Bedding was changed a minimum of once per week. Cages, tops, bottles, etc. were washed with a commercial detergent and allowed to air dry. Prior to use, these items were wrapped and autoclaved. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity were recorded during the study, and the records retained.

### Diet

Animals were fed with sterile Labdiet® 5053 (pre-sterilized) rodent chow and sterile water was provided ad libitum.

### Animal Randomization and Allocations

Mice were randomly and prospectively divided into four treatment groups (Table 1) prior to the initiation of treatment. Each animal was identified by ear punching corresponding to an individual number. A cage card was used to identify each cage and marked with the study number, treatment group number and animal numbers.

### Assessment of Results

Statistical differences between treatment groups were determined using Student's t-test, Mann-Whitney U test and chi-square analysis with a critical value of 0.05.

### Experimental Procedures

Tumours were measured once every two days with micro-calipers, and tumour volume was calculated as (length×width)³π/3. The tumour growth index (TGI) was calculated using the formula 100−(Vc*100/Vt), where Vc is the mean volume of the tumours in the control group and Vt is the mean volume of the tumours in the test group.

### Results

### Survival

No deaths occurred during this study.

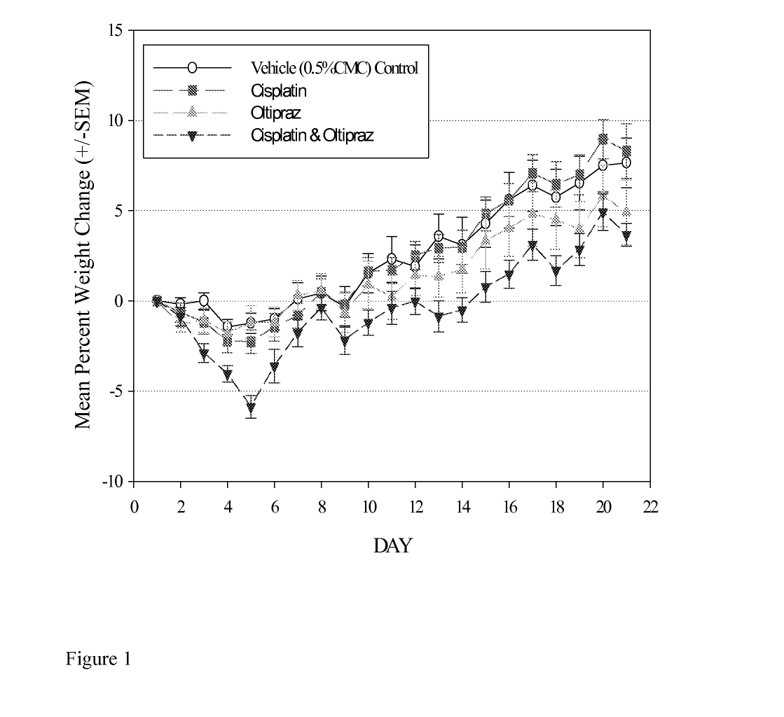

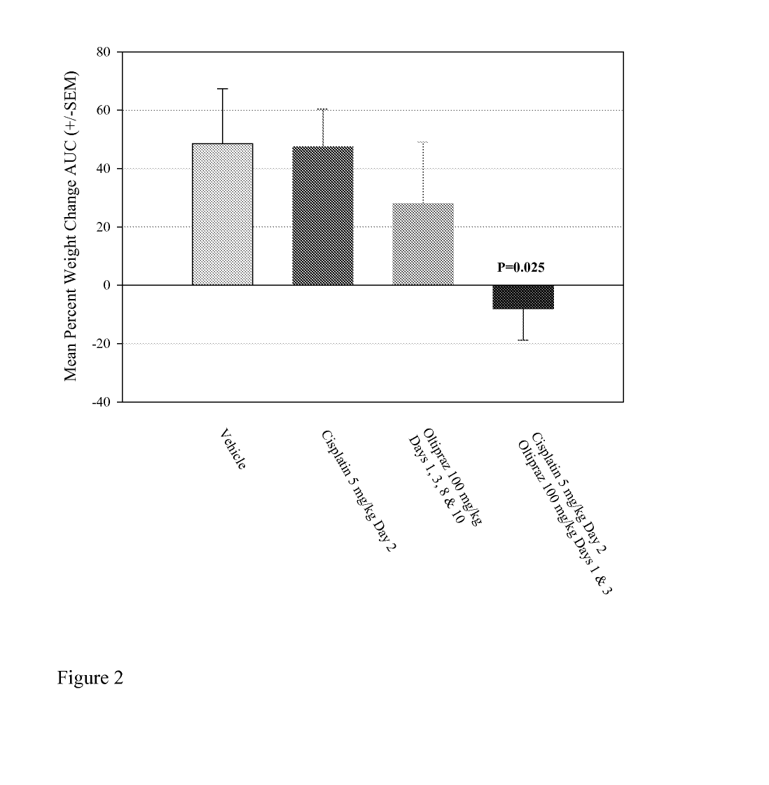

### Animal Weights (FIGS. 1 and 2)

The mean daily percentage weight change for each treatment group is shown in FIG. 1. The mice in the vehicle control group gained an average of 7.6% of their starting weight by day 21. The mice treated with cisplatin 5 mg/kg on day 2 gained an average of 8.3% of their starting weight by day 21. The mice treated with oltipraz 100 mg/kg on days 1, 3, 8 and 10 gained an average of 4.9% of their starting weight by day 21. The mice treated with cisplatin 5 mg/kg on day 2 and oltipraz 100 mg/kg on days 1 and 3 gained an average of 3.7% of their starting weight by day 21.

The significance of these differences was evaluated by calculating the mean area under the curve (AUC) for the percentage weight change for each animal and comparing the groups using an ANOVA on ranks test, with Dunn's post test. The One-Way ANOVA test was not used on this data because the data failed a normality test, indicating that the ANOVA on ranks test was more appropriate for the data set. There was a significant difference between the group treated with cisplatin plus oltipraz group and the vehicle control group (P=0.025). The AUC data is shown in FIG. 2. The AUC calculation was made using the trapezoidal rule transformation. Group means were calculated and are shown with error bars representing SEM for each group. Groups were compared using the ANOVA on ranks method. Statistically significant differences were seen between cisplatin plus oltipraz treated groups and control groups (P=0.025).

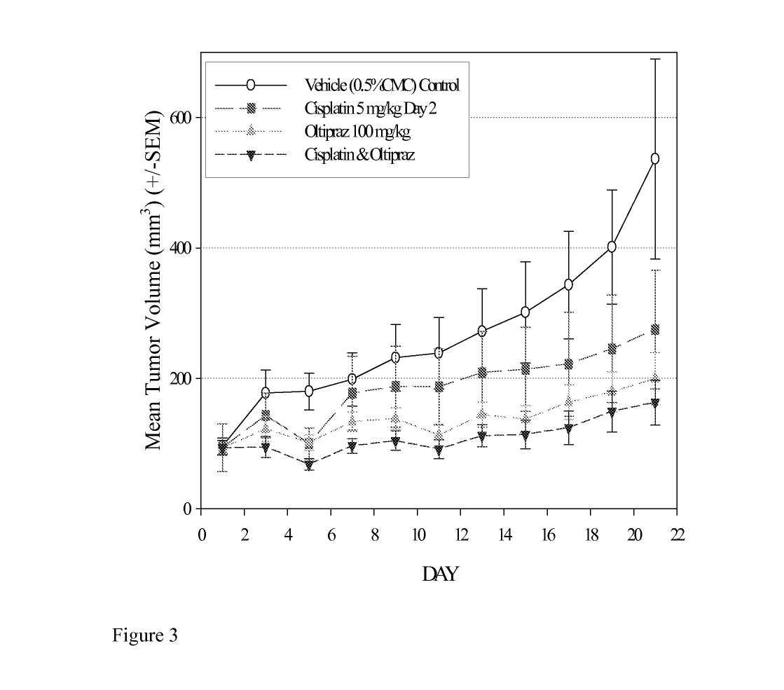

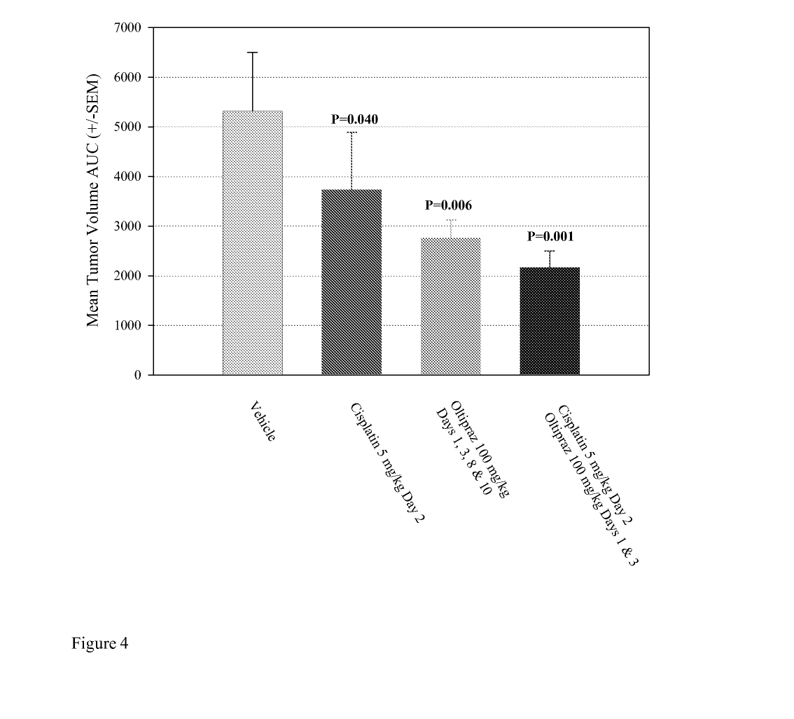

### Tumour Volumes (FIGS. 3 and 4)

Tumour volumes were calculated from the length and width measurements taken on alternating days by calculating the mean radius (r), which was the sum of length and width divided by 4, and using the formula 4/3 πr³ to calculate the volume. The mean tumour volume data is shown in FIG. 3. The mean tumour volume for the vehicle control group increased from 95 mm³ on day 1 to 536 mm³ on day 21. The mean tumour volume for the group treated with cisplatin at 5 mg/kg on day 2 increased from 93 mm³ on day 1 to 275 mm³ on day 21. The mean tumour volume for the group treated with oltipraz at 100 mg/kg on days 1, 3, 8 and 10 increased from 93 mm³ on day 1 to 200 mm³ on day 21. The mean tumour volume for the group treated with cisplatin at 5 mg/kg on day 2 plus oltipraz at 100 mg/kg on days land 3 increased from 93 mm³ on day 1 to 163 mm³ on day 21.

Further analysis of the data was performed by calculating the mean area under the curve (AUC) for the tumour volume for each animal (FIG. 4) and comparing the groups using an ANOVA on ranks test, with Dunn's post test. The area under the curve (AUC) calculation was made using the trapezoidal rule transformation. Group means were calculated and are shown in FIG. 4 with error bars representing SEM for each group. Groups were compared using the ANOVA on ranks method. Statistically significant differences were seen between the vehicle control group and the treatment group (P=0.006). Further comparison of individual groups to the control using the Mann-Whitney rank sum test showed that all three treatment groups had significant reduced tumour growth, with the P values shown in FIG. 4.

The One-Way ANOVA test was not used on this data because the data failed a normality test, indicating that the ANOVA on ranks test was more appropriate for the data set. This analysis indicated that there were significant differences between the treated groups and the vehicle control group (P=0.006), but did not generate P values for individual groups. Individual comparisons of the treated groups to the vehicle control group showed a significant difference using a Rank Sum test between the cisplatin treated group and the vehicle control group (P=0.040), the group treated with oltipraz and the vehicle control group (P=0.006) and the group treated with both cisplatin and oltipraz and the vehicle control group (P=0.001). The Tumour Growth Inhibition (TGI) was calculated for both the cisplatin, oltipraz and the cisplatin plus oltipraz treated groups. The TGI for the group treated with cisplatin alone was 48.8%, and for the group treated with cisplatin plus oltipraz, the TGI was 69.6%. The TGI for oltipraz alone was 62.3%. There was no indication from these data that oltipraz was in any way protective of the A549 tumours. On the contrary, these data indicate that oltipraz was having a significant anti-tumour effect, which was increased when oltipraz was combined with cisplatin. The anti-tumour effect observed for the oltipraz plus cisplatin treatment group was greater than that observed with either oltipraz alone or cisplatin alone.

### Conclusion

Treatment with oltipraz alone showed no evidence of toxicity in this study based on observations of survival and weight change. Treatment with oltipraz in combination with cisplatin caused a statistically significant reduction in weight gain (P=0.025). Tumours in animals treated with a combination of cisplatin and oltipraz grew more slowly than tumours in animals treated with vehicle (P=0.001) and had a TGI of 69.6%. Tumours in animals treated with a combination of cisplatin and oltipraz also grew more slowly than tumours in animals treated with either oltipraz alone or cisplatin alone. There was no evidence that oltipraz negatively interfered with the anti-tumour action of cisplatin in this study. Further, there was no evidence in this study that oltipraz protected the tumours.

### Example 2

### An Evaluation of Oltipraz in Combination with Cetuximab for Efficacy in the Reduction of Tumour Growth

A study was carried out to evaluate the effects of oltipraz on tumour growth when used in conjunction with Cetuximab in the A549 lung cancer model in mice. A549 was used in this study due to its reported sensitivity to Cetuximab.

### Materials and Methods

### Treatment Groups

Thirty-six female nude mice (nu/nu) were randomly assigned into three treatment groups. Each mouse was inoculated with 1×10⁶A549 lung cancer cells in a volume of 0.05 mL on their lower left flank with 0.05 mL of Matrigel. The groups were treated with vehicle (0.5% carboxymethyl cellulose (CMC)), Cetuximab and Cetuximab plus oltipraz as detailed in Table 2. Initiation of treatment was designated day 1.

TABLE 2
Treatment Groups.

| | | | | Drug | | |
|---|---|---|---|---|---|---|
| | No. | Tumour cell | Adjuvant | Treatment | | Dose |
| Group | of animals | inoculum | Therapy | & Dosing | Route | Sched. |
| 1 | 12 | 1 × 10⁶ | none | Vehicle | po | qd, |
| | | | | (0.5% CMC) | | days 1, 3, |
| | | | | | | 8 & 10 |
| 2 | 12 | 1 × 10⁶ | Cetuximab IV | Vehicle | po | qd, |
| | | | 1.7 mg/kg Day 2 | (0.5% CMC) | | days 1, 3, |
| | | | Cetuximab IV | | | 8 & 10 |
| | | | 1.0 mg/kg Day 9 | | | |
| 3 | 12 | 1 × 10⁶ | Cetuximab IV | Oltipraz | po | qd, |
| | | | 1.7 mg/kg Day 2 | 100 mg/kg | | days 1, 3, |
| | | | Cetuximab IV | | | 8 & 10 |
| | | | 1.0 mg/kg Day 9 | | | |

### Weights and Survival

All animals were weighed every day and their survival recorded, in order to assess possible differences in animal weight among treatment groups as an indication of possible toxicity resulting from the treatments. Any animals exhibiting a loss of >20% of starting weight during the course of the study were euthanized.

### Tissue Culture

A549 human lung cancer cells were obtained from ATCC. These cells were grown in F-12 supplemented with 10% Fetal Calf Serum (FCS), penicillin and streptomycin, and 2 mM L-Glutamine. Cells were sub-cultured by removing the medium, rinsing twice with sterile calcium-free and magnesium-free phosphate buffered saline (PBS) and adding 1 to 2 ml of 0.25% trypsin and 0.03% EDTA solution. The flask was allowed to sit at 37° C. until the cells detached. Cells were then sub-cultured at a ratio of 1:3.

### Animals

Female Nude Mice which are homozygous for the nu gene (nu+/nu+) (Charles River Labs), aged 5 to 6 weeks, with a mean pre-treatment body weight of 24.6 grams were used. Animals were individually numbered using an ear punch and housed in groups of 12 animals per cage. Animals were acclimatized prior to study commencement. During this period of at least 2 days, the animals were observed daily in order to reject animals that presented in poor condition.

### Housing

The study was performed in animal rooms provided with filtered air at a temperature of 70° F.+/−5° F. and 50%+/−20% relative humidity. Animal rooms were set to maintain a minimum of 12 to 15 air changes per hour. The room was on an automatic timer for a light/dark cycle of 12 hours on and 12 hours off with no twilight. Sterilized Bed-O-Cobs® bedding was used. Bedding was changed a minimum of once per week. Cages, tops, bottles, etc. were washed with a commercial detergent and allowed to air dry. Prior to use, these items were wrapped and autoclaved. A commercial disinfectant was used to disinfect surfaces and materials introduced into the hood. Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number and treatment group marked all cages. The temperature and relative humidity were recorded during the study, and the records retained.

### Diet

Animals were fed with sterile Labdiet® 5053 (pre-sterilized) rodent chow and sterile water was provided ad libitum.

### Animal Randomization and Allocations

Mice were randomly and prospectively divided into three treatment groups prior to the initiation of treatment. Each animal was identified by ear punching corresponding to an individual number. A cage card was used to identify each cage and marked with the study number, treatment group number and animal numbers.

### Assessment of Results

Statistical differences between treatment groups were determined using Student's t-test, Mann-Whitney U test and chi-square analysis with a critical value of 0.05.

### Experimental Procedures

Tumours were measured once every two days with micro-calipers, and tumour volume was calculated as (length×width)³π/3. The tumour growth index (TGI) was calculated using the formula 100−(Vc*100/Vt), where Vc is the mean volume of the tumours in the control group and Vt is the mean volume of the tumours in the test group.

### Results

### Survival

One animal died in this study on day 21 in the Cetuximab only treated group. The reason for the death of this animal is unclear. The tumour burden was relatively high but there was no evidence of significant weight loss.

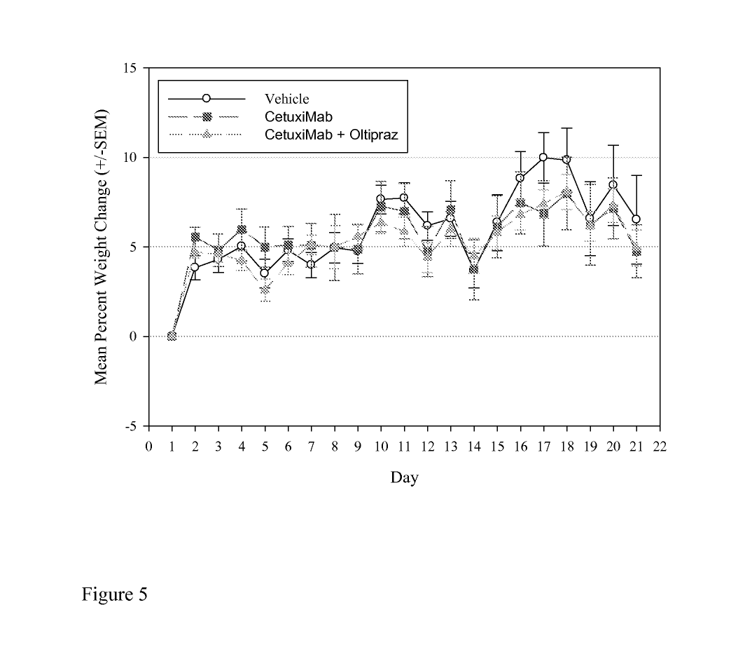

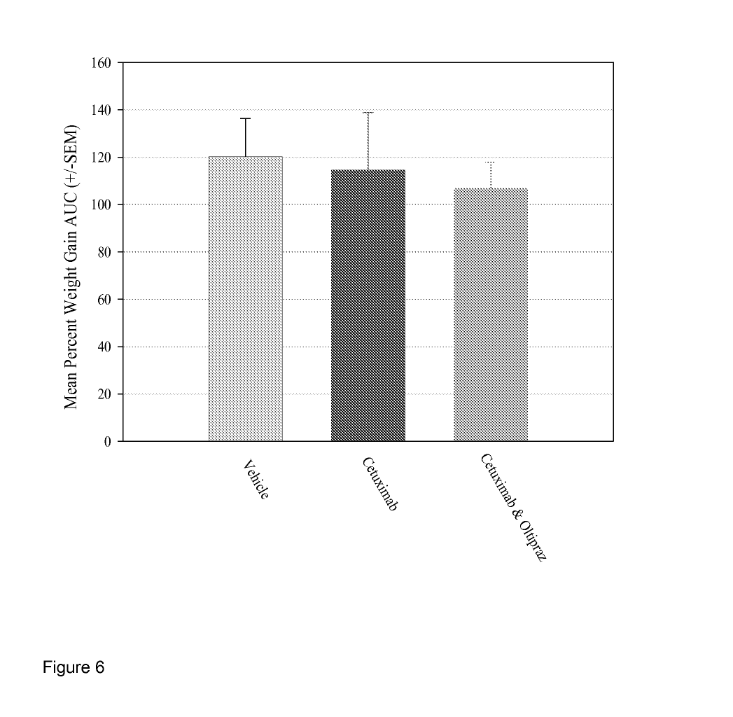

### Animal Weights (FIGS. 5 and 6)

The mean daily percentage weight change for each treatment group is shown in FIG. 5. The mice in the vehicle control group gained an average of 6.5% of their starting weight by day 21. The mice treated with Cetuximab 1.7 mg/kg on day 2 and 1.0 mg/kg on day 9 gained an average of 4.8% of their starting weight by day 21. The mice treated with Cetuximab 1.7 mg/kg on day 2 and 1.0 mg/kg on day 9 and oltipraz 100 mg/kg on days 1, 3, 8 and 10 gained an average of 4.9% of their starting weight by day 21.

The significance of these differences was evaluated by calculating the mean area under the curve (AUC) for the percentage weight change for each animal and comparing the groups using a One-Way ANOVA test. There were no significant differences between the oltipraz treated groups and the vehicle control groups (P=0.828). The AUC data is shown in FIG. 6. This calculation was made using the trapezoidal rule transformation. Group means were calculated and are shown with error bars representing SEM for each group. Groups were compared using the One-Way ANOVA method. No statistically significant differences were seen between oltipraz treated and control groups (P=0.828).

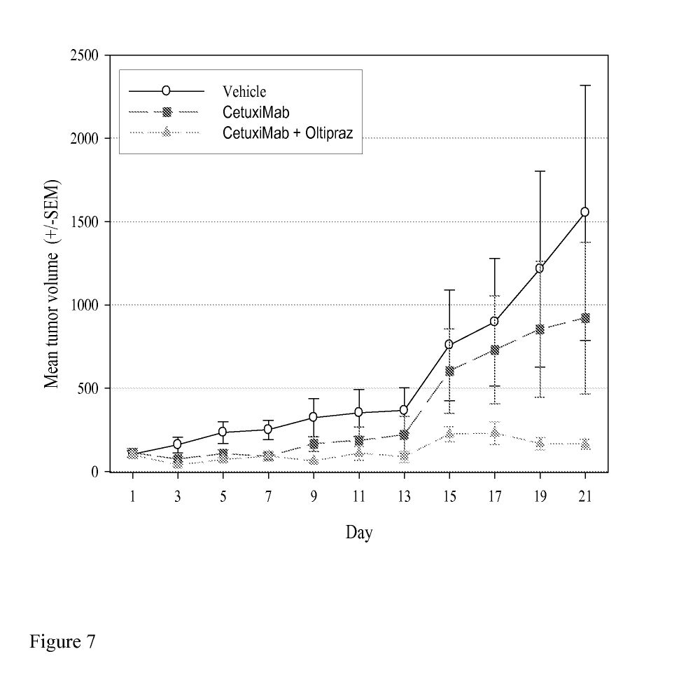

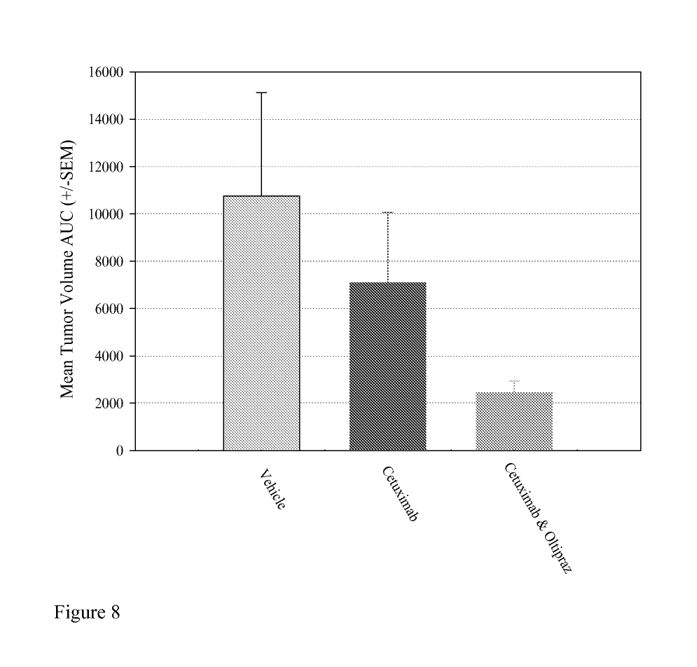

### Tumour Volumes (FIGS. 7 and 8)

Tumour volumes were calculated from the length and width measurements taken on alternating days by calculating the mean radius (r), which was the sum of length and width divided by 4, and using the formula 4/3 πr³ to calculate the volume. The mean tumour volume data is shown in FIG. 7. The mean tumour volume for the vehicle control group increased from 103 mm³ on day 1 to 1553 mm³ on day 21. The mean tumour volume for the group treated with Cetuximab at 1.7 mg/kg on day 2 and 1.0 mg/kg on day 9 increased from 112 mm³ on day 1 to 607 mm³ on day 21. The mean tumour volume for the group treated with Cetuximab at 1.7 mg/kg on day 2 and 1.0 mg/kg on day 9 plus oltipraz at 100 mg/kg on days 1, 3, 8 and 10 increased from 99 mm³ on day 1 to 164 mm³ on day 21.

Further analysis of the data was performed by calculating the mean area under the curve (AUC) for the tumour volume for each animal (FIG. 8). This calculation was made using the trapezoidal rule transformation. Group means were calculated and are shown with error bars representing SEM for each group. Groups were compared using the One-Way ANOVA method. This analysis did not reveal significant differences between either of the treated groups and the vehicle control group (P=0.176). Individual comparisons of the treated groups to the vehicle control group showed no significant differences using a Rank Sum test between either the Cetuximab treated group and the vehicle control group (P=0.583) or the group treated with Cetuximab and oltipraz and the vehicle treated control group (P=0.078). There were no statistically significant differences between the Cetuximab treated group and the Cetuximab plus oltipraz treated group (P=0.214).

The comparison between the vehicle treated group and the other two groups was characterized by an unusually high degree of variation in the control group. Animal #2 in particular had the smallest tumour in the study on day 21. If the data from this animal is excluded, the group treated with Cetuximab plus oltipraz shows a significant reduction in tumour volume AUC (P=0.025). This suggests a synergistic effect of Cetuximab and oltipraz which resulted in decreased tumour volume.

The Tumour Growth Inhibition (TGI) was calculated for both the Cetuximab and the Cetuximab plus Oltipraz treated groups. The TGI for the group treated with Cetuximab alone was 61%, and for the group treated with Cetuximab plus oltipraz, the TGI was 89.4%. There was no indication from these data that oltipraz was in any way protective of the A549 tumours.

### Conclusions

Oltipraz showed no evidence of toxicity in this study based on observations of survival and weight change. Tumours in animals treated with Cetuximab grew more slowly than tumours in animals treated with vehicle and had a TGI of 61%. Tumours in animals treated with Cetuximab and oltipraz grew more slowly than tumours in animals treated with vehicle and had a TGI of 89.4%. There was no evidence that oltipraz negatively interfered with the anti-tumour action of Cetuximab in this study. There was no evidence in this study that oltipraz protected the tumours.

Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

1. A method for the prophylaxis and/or treatment of a tumour in a subject, the method comprising the steps of:
administering a therapeutically effective amount of a composition comprising 5-(2-pyrazinyl)-4-methyl-1,2-dithiol-3-thione, or an analogue, derivative, metabolite, prodrug, solvate or pharmaceutically acceptable salt thereof; and
administering a therapeutically effective amount of a composition comprising an anti-tumour agent to the subject.

2. (canceled)

3. A method as claimed in claim 1, wherein the anti-tumour agent is a platinum-based agent.

4. A method as claimed in claim 3, wherein the platinum-based agent is selected from the group consisting of cisplatin, carboplatin and oxaliplatin.

5. A method as claimed in claim 4, wherein the platinum-based agent is cisplatin.

6. A method as claimed in claim 1, wherein the anti-tumour agent is a monoclonal antibody.

7. (canceled)

8. A method as claimed in claim 6, wherein the monoclonal antibody is Cetuximab.

9-11. (canceled)

12. A method as claimed in claim 1, wherein 5 (2 pyrazinyl)-4-methyl-1,2-dithiol-3-thione, or the analogue, derivative, metabolite, prodrug, solvate or pharmaceutically acceptable salt thereof is formulated in carboxymethyl cellulose.

13. (canceled)

14. A method as claimed in claim 1 further comprising the step of administering cysteine or an analogue, derivative, salt or solvate thereof.

15. A composition comprising an anti-tumour agent and 5-(2-pyrazinyl)-4-methyl-1,2-dithiol-3-thione, or an analogue, derivative, metabolite, prodrug, solvate or pharmaceutically acceptable salt thereof.

16. A composition as claimed in claim 15, wherein the anti-tumour agent is a platinium-based agent.

17. A composition as claimed in claim 16, wherein the platinum-based agent is selected from the group consisting of cisplatin, carboplatin and oxaliplatin.

18. A composition as claimed in claim 17, wherein the platinum-based agent is cisplatin.

19. A composition as claimed in claim 15, wherein the anti-tumour agent is a monoclonal antibody.

20. (canceled)

21. A composition as claimed in claim 19, wherein the monoclonal antibody is Cetuximab.

22. A composition as claimed in claim 15, further comprising carboxymethyl cellulose.

23. (canceled)

24. A composition as claimed in claim 15, further comprising cysteine or an analogue, derivative, salt or solvate thereof.

25. A composition as claimed in claim 15, comprising at least one pharmaceutically acceptable carrier, excipient or diluent.

26-30. (canceled)

31. A method for reducing the toxicity of an anti-tumour agent in a subject, the method comprising the steps of:
administering a therapeutically effective amount of a composition comprising 5-(2-pyrazinyl)-4-methyl-1,2-dithiol-3-thione, or an analogue, derivative, metabolite, prodrug, solvate or pharmaceutically acceptable salt thereof; and
administering a therapeutically effective amount of a composition comprising the anti-tumour agent to the subject.

32. A method as claimed in claim 31, wherein the anti-tumour agent is a platinum-based agent.

33. A method as claimed in claim 32, wherein the platinum-based agent is selected from the group consisting of cisplatin, carboplatin and oxaliplatin.

34. A method as claimed in claim 33, wherein the platinum-based agent is cisplatin.

35. A method as claimed in claim 31, wherein the anti-tumour agent is a monoclonal antibody.

36. A method as claimed in claim 35, wherein the monoclonal antibody is Cetuximab.

37. A method as claimed in claim 31, wherein 5-(2-pyrazinyl)-4-methyl-1,2-dithiol-3-thione, or the analogue, derivative, metabolite, prodrug, solvate or pharmaceutically acceptable salt thereof is formulated in carboxymethyl cellulose.

38. A method as claimed in claim 31, further comprising the step of administering cysteine or an analogue, derivative, salt or solvate thereof.
